(19) United States
(12) Patent Application Publication
(10) Pub. No.: US 2008/0200680 A1
(43) Pub. Date: Aug. 21, 2008

(54) Polymorph Transformation of Zolpidem in Tablet Matrix

(76) Inventors: Brian K. Cheng, Chesterfield, MO (US); Stephen H. Wu, Chesterfield, MO (US)

Correspondence Address: Mallinckrodt Inc., 675 McDonnell Boulevard, HAZELWOOD, MO, 63042, US

(21) Appl. No.: 12/089,929

(22) PCT Filed: Sep. 28, 2006
(86) PCT No.: PCT/US06/37586

§ 371 Date: Apr. 11, 2008

Related U.S. Application Data
(60) Provisional application No. 60/727,371, filed on Oct. 17, 2005.

(51) Int. Cl. C07D 471/04

(52) U.S. Cl. 546/121

(57) ABSTRACT

A method for converting polymorphs of Zolpidem hemitartrate, the method comprising treating tablets comprising Zolpidem hemitartrate with heat and/or moisture.

### FIELD OF THE INVENTION

The present invention relates to methods for preparing solid dose preparations comprising Zolpidem hemitartrate, and more particularly the invention relates to converting Zolpidem hemitartrate polymorphs into a desired polymorph in the process of making tablets.

### BACKGROUND OF THE INVENTION

Zolpidem, a known pharmaceutical that possesses anxiolytic, sedative, and hypnotic properties and which is F.D.A. approved for short-term treatment of insomnia, has the following structural formula:

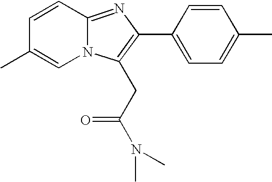

Many pharmaceutical solids, including Zolpidem, exist in different physical forms, e.g., crystalline or amorphous. Polymorphism refers to the occurrence of different crystalline forms of the same drug substance. Amorphous solids consist of disordered arrangements of molecules and do not possess a distinguishable crystal lattice. Solvates are crystalline solids containing amounts of a solvent incorporated within the crystal structure. If the incorporated solvent is water, the solvates are also commonly known as hydrates.

It is known in the art that the crystal forms (polymorphs) of a drug molecule can be made or transformed under different environmental conditions, typically in contact with water, organic solvents, mixtures of solvents, or vapors of solvents. Polymorphs and/or solvates of a drug molecule may have different chemical and/or physical properties. For example, polymorphs and/or solvates can differ substantially in melting point, chemical reactivity, particle size, shape, flow characteristics, caking, degree of hydration or salvation, optical and electrical properties, vapor pressure, and density. As a result, certain polymorphs of a drug molecule are more stable in a given environmental condition or selected solvent system than others.

A number of methods have been employed for characterizing polymorphs in pharmaceutical solids (H. Brittain. Methods for the Characterization of Polymorphs and Solvates, POLYMORPHISM IN PHARMACEUTICAL SOLIDS, H. G. Brittain (ed.), Marcel Dekker, Inc., New York, 1999, pp. 227-278). Among the most common are polarizing optical microscopy and thermomicroscopy, thermal analysis procedures, such as differential scanning calorimetry (DSC) and thermogravimetric analysis (TGA), and solid-state spectroscopy. While the definitive criterion for the existence of polymorphism is via demonstration of a nonequivalent crystal structure, usually by comparison of the x-ray diffraction patterns (such as by powder X-ray diffraction (pXRD)), supporting information such as microscopy, thermal analysis methodology, and solid state NMR are commonly used.

Polymorphism has a direct impact on the processability of drug substances and the quality of the final product. For example, physical properties including particle size, shape, flow characteristics, melting point, degree of hydration or solvation, and caking tendency can cause difficulties in chemical processing, material handling, compatibility with excipients, segregation in the blend, dissolution rate of a drug in aqueous media, and stability of the final dosage form. Whereas a change in chemical properties due to polymorph transformation can affect drug degradation induced by environmental factors such as heat, light, moisture, mechanical handling, oxygen, and interaction with excipients. The adverse effects may cause loss of production efficiency (time and cost), product quality and instability. Thus, it is desirable to utilize a proper polymorph in developing the dosage form.

The most stable polymorph of a drug substance is often used because it has the lowest potential for conversion from one polymorph to another, while a metastable polymorph may be used to enhance bioavailability. Gibbs free energy, thermodynamic activity, and solubility provide the definitive measures of relative polymorphic stability under defined conditions of temperature and pressure.

One polymorph may convert to another during manufacturing and storage, particularly when a metastable polymorph is used. Since an amorphous form is thermodynamically less stable than any crystalline form, inadvertent crystallization from an amorphous drug substance may occur. Because of the higher mobility and ability to interact with moisture, amorphous drug substances are also more likely to undergo solid-state reactions. Solid-state reactions include solid-state phase transformations, dehydration/desolvation processes, and chemical reactions.

In addition, phase conversions of some drug substances are possible when exposed to a range of manufacturing processes (H. G. Brittain and E. F. Fiese, Effect of Pharmaceutical Processing on Drug Polymorphs and Solvates, POLYMORPHISM IN PHARMACEUTICAL SOLIDS, H. G. Brittain (ed.), Marcel Dekker, Inc., New York, 1999, pp. 331-362). Milling operations may result in polymorphic conversion of a drug substance. In the case of wet granulation processes, where the usual solvents are aqueous, one may encounter a variety of conversions between anhydrates and hydrates, or between different hydrates.

It is advantageous to have a method, which specifically can convert one polymorph or a mixture of polymorphs or a mixture of polymorphs and amorphous material to a desirable polymorph in the final dosage form during the formulation process. Benefits include simplifying the process steps, reducing manufacturing costs, and increasing processing ease for both the pharmaceutical active ingredient and the finished dosage form.

Zolpidem hemitartrate is known to exist in several polymorphs, among which are known the A, B, C, D, E, F, G, and H forms. See WO 01/80857 A1 by Teva Pharmaceutical Industries, Ltd. Teva Pharmaceutical Industries, Ltd. disclosed a method for converting Zolpidem polymorphs by solvating with water, methanol, ethanol, propanol, butanol, ethyl acetate, and the like. The results from the disclosed method often are irreproducible, particularly in production scale. In the disclosure, polymorph E was converted from other polymorphs isolated from water or solvent contact. The extra chemical processing steps and the need for solvent recovery steps required in the method can increase the production cost. Furthermore, some polymorphs are particularly difficult to process because of their physical properties.

Zolpidem hemitartrate may also undergo polymorph transformation under ambient storage conditions. We found Zolpidem polymorph E in the innovator's products according to the result of pXRD analysis; however, the starting material should be polymorph A according to the monograph of European Pharmacopoeia. It is desirable to have a consistent polymorph E in the finished product to provide the consistent release profile and bioavailability.

### SUMMARY OF THE INVENTION

Among the various aspects of the present invention is a method for polymorph transformation of Zolpidem hemitartrate in a tablet matrix in the dosage formulation process. The conversion of polymorphs in a tablet during the dosage formulation process eliminates the need for a chemical process to produce a desirable form prior to formulating the active into the final dosage form.

Another aspect of the present invention is a method for polymorph transformation of Zolpidem hemitartrate in the process for coating substrates such as tablets or particles. In one embodiment, the transformation consists of converting polymorphs of Zolpidem hemitartrate to a stable polymorph in the spray-dried process.

Briefly, therefore, the invention is directed to a method for converting Zolpidem hemitartrate salt to a desired polymorph of Zolpidem hemitartrate salt comprising preparing a tablet comprising Zolpidem hemitartrate salt and solvating the tablet with an amount of a solvent to convert Zolpidem hemitartrate salt to the desired polymorph of Zolpidem hemitartrate salt.

The invention is further directed to a method for converting Zolpidem hemitartrate salt to a desired polymorph of Zolpidem hemitartrate salt comprising preparing a tablet comprising the hemitartrate salt of the compound and heating the tablet to convert the hemitartrate salt of the compound to the desired polymorph of the hemitartrate salt of the compound.

The invention is still further directed to a method for converting Zolpidem hemitartrate salt to a desired polymorph of Zolpidem hemitartrate salt comprising preparing a coating solution comprising Zolpidem hemitartrate salt and coating a tablet with the coating solution to convert Zolpidem hemitartrate salt to the desired polymorph of Zolpidem hemitartrate salt.

Other aspects of the invention are described in more detail below.

### DETAILED DESCRIPTION OF THE PREFERRED EMBODIMENT(S)

The present invention describes methods for transforming Zolpidem hemitartrate present in a variety of polymorphs into a desirable polymorph during the dosage formulation process to simplify the overall process, reduce production cost, and improve the product quality. In particular, the invention comprises a method for converting various polymorphs of Zolpidem hemitartrate or amorphous material to a desired polymorph in the tablet matrix. Note that the Zolpidem polymorphs discussed herein are those identified in WO 01/80857 A1 by Teva Pharmaceutical Industries, Ltd., the disclosure of which is hereby incorporated by reference in its entirety for all purposes.

In accordance with the invention, Zolpidem hemitartrate in any of its polymorphs or as a mixture of polymorphs or as an amorphous material is mixed with suitable pharmaceutical excipients to form a tablet comprising Zolpidem hemitartrate, which is then subjected to further treatment to convert the various polymorphs into a desired polymorph, such as, for example, polymorph C, polymorph D, or preferably into polymorph E. Typical pharmaceutical excipients include sugars such as lactose, fructose, maltodextrin, maltose, mannitol, sorbitol, sucrose, and mixtures thereof; organic acids including citric acid, tartaric acid, glycolic acid, and mixtures thereof; buffers including acetate, citrate, tartrate, oxalate, phosphate, carbonate, and mixtures thereof; polymeric materials including microcrystalline cellulose (MCC, Avicel®, available from FMC Corporation), hydroxymethylpropyl cellulose (HMPC, Opadry®, available from Colorcon), ethyl cellulose, propyl cellulose, starch, sodium starch glycolate, and mixtures thereof; and lubricants such as magnesium stearate. Preferably, the excipients are chosen to facilitate polymorph transformation. More preferably, the pharmaceutical excipients include lactose, magnesium stearate, and microcrystalline cellulose and/or sodium starch glycolate. The Zolpidem hemitartrate polymorphs and pharmaceutical carriers can be dry blended and compressed into tablets according to methods known in the art. A compressed tablets typically weighs about 120 mg and comprises between about 5 mg and about 10 mg Zolpidem hemitartrate.

Polymorphic transformations can be carried out by subjecting tablets comprising Zolpidem hemitartrate in any of its polymorphs to heat and/or environmental moisture under a controlled process condition. The tablets can be placed in an environmental chamber in which the temperature, relative humidity, and other conditions can be controlled. For example, the environmental chamber can be an oven which allows temperature and humidity control, and the tablets can be heated to temperatures in excess of about 40° C., preferably at least about 50° C., more preferably at least about 65° C. During heating, the relative humidity can be controlled such that the relative humidity is at least about 50%, preferably at least about 75%. Preferably, the humid atmosphere comprises water vapor. The Zolpidem hemitartrate polymorphs can be converted to desirable stable polymorphs according to these conditions within the tablet matrix. For example, it has been discovered that a heat treatment under relatively dry conditions can be used to convert Zolpidem hemitartrate polymorphs preferably to polymorph C. Under a heat and humidity treatment, the Zolpidem hemitartrate polymorphs preferably convert to polymorph D.

The Zolpidem hemitartrate polymorphs within the tablet matrix can be converted to polymorph E by high moisture or wetting treatment with controlled drying. The wetting treatment can occur by spraying or immersion with the condition that the treatment achieves sufficient wetting throughout the entire tablet without compromising tablet integrity. To achieve sufficient wetting, process conditions such as water flow rate, air flow, and drying temperature are balanced and optimized depending upon the size of the pan coater or other equipment, the batch size, the tablet shape, and tablet hardness. Although precise values for water flow rate, air flow, and drying temperature vary as a function of the above named parameters, it is important that they be balanced to sufficiently wet the tablets to allow moisture to disperse throughout the tablet with simultaneous drying. Preferably, the tablets are wetted with water. In an exemplary wetting process, the tablets are charged to a pan coater, rotated, and wetted by spraying with water under conditions of moderate heating and air flow rate. The tablets are preferably coated with HMPC, marketed as Opadry®, to harden the tablets and also increase the tablets' hygroscopicity. Preferably, process conditions are optimized to allow the tablets to absorb at least about 5% by wt. water for sufficient wetting to convert the Zolpidem hemitartrate polymorphs to polymorph E without compromising the integrity of the tablet. It has been discovered that wetting at a temperature between about 25° C. and about 45° C. with an inlet air flow of about 22 CFM and a pan speed of about 10 rpm is sufficient to allow the tablets to absorb about 5% by wt. water.

It has also been discovered that wetted tablets comprising polymorph E, preferably prepared according to the above-described method, can be heat treated to a temperature of at least about 50° C., more preferably at least about 80° C. to convert the polymorph E to a different polymorph, for example, polymorph C.

Alternatively, polymorphic transformations can be carried out by spraying placebo tablets with a solution or dispersion comprising Zolpidem hemitartrate in any of its polymorphs or as a mixture of polymorphs or as an amorphous material during a coating process. As a result of the slurry preparation and coating process, the Zolpidem hemitartrate polymorphs are substantially converted to polymorph E in the coated tablet.

To prepare the coating solution, Zolpidem hemitartrate is dissolved or suspended in water, aqueous solution, or a mixture of water and a minor amount of pharmaceutically acceptable solvent such as methanol, ethanol, propanol, butanol, or ethyl acetate. Preferably, the solvent is water. The aqueous solution comprising Zolpidem hemitartrate may also comprise polymeric binders, such as Opadry®.

Substrates useful for coating with the Zolpidem hemitartrate solution are preferably pills or tablets comprising pharmaceutical excipients commonly used in making tablets or particles for solid dosage forms. Such excipients include those listed above. Preferably, the placebo tablet comprises lactose, microcrystalline cellulose, and magnesium stearate. Another preferred placebo tablet formulation comprises lactose, microcrystalline cellulose, magnesium stearate, hydroxymethylpropylcellulose, and sodium starch glycoate. Preferably, the substrates suitable for coating exhibit sufficient integrity and water-absorbing capacity when water, aqueous solution, or a mixture of solvents is applied to it.

The aqueous solution of Zolpidem hemitartrate, or the suspension of Zolpidem hemitartrate in a suitable solvent system, can be applied to the substrates using conventional spray coating equipment such as a pan coater or a fluid bed coater. It has been discovered that spray coating placebo tablets with a solution comprising Zolpidem hemitartrate yields active tablets comprising Zolpidem hemitartrate polymorph E.

The following examples further illustrate the practice of the present invention.

### Example 1

### Tablet Preparation

Zolpidem hemitartrate (Polymorph A), lactose, and magnesium stearate were thoroughly mixed in a beaker. The amount of each component is shown in Table 1. The powder blend was fed into a tablet press (Korsch PHI 06) and compacted to make tablets using 0.3437 inch deep cup round toolings. Each tablet weighed about 120 mg, and had a hardness value about 10 kPa.

TABLE 1
Zolpidem Hemitartrate Form A Tablets Composition

| Material | Wt (g) | Wt. % |
|---|---|---|
| Zolpidem hemitartrate Polymorph A | 1 | 8% |
| Lactose 316 (Farmost) | 10.94 | 91% |
| Magnesium stearate | 0.06 | 1% |
| Total | 12 | 100% |

### Example 2

### Zolpidem Polymorph Conversion by Heat and Moisture Treatment

A sample of the tablets of Example 1 was heated in an oven at 65° C. for 18 hours. Another tablet sample was heated in a humidity-controlled oven (75% Relative Humidity, 50° C., 24 hours). The above treated samples, as well as untreated powder blend and untreated tablets from Example 1, were analyzed by powder x-ray diffraction (pXRD).

Results from pXRD analysis indicated that the Zolpidem hemitartrate in the untreated powder blend and untreated tablets remained as polymorph A. The Zolpidem hemitartrate in the tablets treated at 65° C. transitioned into polymorph C. The Zolpidem hemitartrate in the tablets treated with heat and humidity transitioned into polymorph D. The results are shown in Table 2.

TABLE 2
Result from Thermal Treatment

| Starting Material | Treatment | Polymorph |
|---|---|---|
| Powder blend (Polymorph A) | None | A |
| Tablets (Polymorph A) | Compressed into tablets | A |
| Tablets (Polymorph A) | Heated at 65° C. | C |
| | in an oven for 18 hours | |
| Tablets (Polymorph A) | Heated at 50° C./75% | D |
| | RH for 24 hours | |

### Example 3

### Zolpidem Polymorph Conversion from Polymorph a to Polymorph E by Water Treatment

Tablets containing Zolpidem hemitartrate polymorph A (Example 1) were treated to convert the Zolpidem hemitartrate polymorph A to stable polymorph E according to the following protocol:
1. Zolpidem hemitartrate polymorph A, Lactose 316, and microcrystalline cellulose (Avicel® PH 200) were charged into a V-shape blender and mixed for 5 minutes. The amount of each component is shown in Table 3a.
2. Magnesium stearate (amount shown in Table 3a) was added to the powder, and the powder mixed for an additional 3 minutes.
3. Powder blend from step 2 was pressed into 120 mg tablets on a tablet press (Manesty Beta tablet press (No. 59348 punches)) using the parameters given in Table 3b. Force Feeder was applied, and the tablets were compressed at 6 kN compression force. The tablet hardness was measured by a hardness tester, and the average hardness value was about 5 kPa.
4. Zolpidem hemitartrate polymorph A tablets from step 3 were charged into a pan coater, which was run using the parameters shown in Table 3c.
5. Water was applied via spray gun to wet the tablet surfaces. When the tablet surfaces were sufficiently wetted, the pan was stopped. The tablets were immediately removed from the coater and sealed in a Ziploc bag.
6. The polymorph form of the tablets was determined by pXRD after 24 hours.

Under a slow spray condition, when the tablets absorbed only 1% by wt. water, there was no detectable polymorph change in the tablets after 24 hours. The water spray procedure was repeated again and the tablet water content was increased to about 5% by weight. The tablet sample was analyzed by pXRD, and the Zolpidem hemitartrate was found to have converted into polymorph E. See Table 3d.

TABLE 3a
Tablet Composition Containing Zolpidem Hemitartrate Form A

| Material | Wt (g) | Wt. % |
|---|---|---|
| Zolpidem hemitartrate | 100 | 3% |
| Lactose 316 | 2288 | 76% |
| Avicel ® PH200 | 600 | 20% |
| Magnesium stearate | 27 | 1% |
| Total | 3015 | 100% |

TABLE 3b
Tableting Parameters

| | |
|---|---|
| Tooling | (0.2795″), deep cup, round |
| Tablet wt (mg) | 120 |
| Hardness | 5 kPa |
| Speed | 80 RPM |

TABLE 3c
Accela Cota Pan Coater Parameters.
Target Process Parameters

| | |
|---|---|
| Inlet Air Temp | 25° C. to 45° C. |
| Exhaust Air | 20° C. |
| Inlet Airflow | 22 CFM |
| Pan Speed | 10 rpm |
| Pump Speed | 5 rpm |
| Pan Charge | 600 g |
| Pump Reverse Time | 99 sec |
| Nozzle Pressure | 11 psi |
| Pan Jog Time | 10 sec |

TABLE 3d
Polymorph Transformation Result by Water Uptake

| Amount of water weight gain | Form |
|---|---|
| 1% | A |
| 5% | E |

### Example 4

### Zolpidem Polymorph Conversion from Polymorph E to Polymorph C by Heat Treatment

Tablets from Example 3 were heated in an oven at 80° C. overnight and analyzed by pXRD. Zolpidem hemitartrate polymorph E in the tablets converted to polymorph C.

### Example 5

### Preparation of Coated Placebo Tablets Having Zolpidem Hemitartrate in Polymorph E

Placebo tablets were prepared having components shown in Table 4a. The tablets were prepared according to the following protocol:
1. Microcrystalline cellulose (Avicel® PH200) and Lactose 316 were charged into a V-shape blender, and the powder was mixed for 5 minutes.
2. Magnesium stearate was added, and the powder mixed for an additional 3 minutes.
3. The powder blend was compressed (Manesty Beta tablet press) into tablets using the parameters given in Table 4b. Force Feeder was used, and the tablets were compressed at 7 kN compression force. The tablets weighed about 120 mg and had a hardness value about 5 kPa.
4. A coating dispersion was prepared containing Zolpidem hemitartrate as polymorph A and Opadry® (Colorcon). The coating composition is shown in Table 4c.
5. The tablets (600 g) were charged into the pan coater and coated using the coating parameters shown in Table 4d. The active suspension was sprayed onto the placebo tablets.

The tablets resulting from Step 5 were shown by pXRD to contain Zolpidem hemitartrate polymorph E.

TABLE 4a
Placebo Tablet Composition

| Component | Wt (g) | Wt. % |
|---|---|---|
| Microcrystalline cellulose (Avicel ® PH200) | 800 | 20% |
| Lactose 316 | 3180 | 79.50% |
| Magnesium stearate | 20 | 0.50% |
| Total | 4000 | 100% |

TABLE 4b
Tableting Parameters

| | |
|---|---|
| Tooling | 0.2795 inch, deep cup, round |
| Tablet wt (mg) | 120 |
| Hardness | 5 kPa |
| Speed | 80 RPM |

TABLE 4c
Active Coating Composition

| Component | Wt. (g) | Note |
|---|---|---|
| Placebo Tablets | 600 | |
| Zolpidem hemitartrate | 27.5 | less than 2% concentration |
| Water | 1375 | |
| Opadry ® | 35 | 2% solution |

TABLE 4d
Coating Process Parameters
Target Process Parameters

| | |
|---|---|
| Inlet Air Temp | 60° C. |
| Exhaust Air | ~35-40° C. |
| Inlet Airflow | 31 CFM |
| Pan Speed | 10 rpm |
| Pump Speed | 10 rpm |
| Pan Charge | 600 g |
| Pump Reverse Time | 99 sec |
| Nozzle Pressure | 11 psi |
| Pan Jog Time | 10 sec |

### Example 6

### Stability Testing of Tablets Coated with Zolpidem Hemitartrate Polymorph E

To test the stability of the Zolpidem hemitartrate polymorph E in the coated tablets of Example 5, the tablets were subjected to the following post-treatment steps
1. A tablet sample (1 kg) from Example 5 was milled using CoMil equipped with approximately 1000 μm grated screen (2A062G03123139) and impeller (2A1601173). The machine was run at 1300 rpm. The product was in granular form.
2. The granules were blended with microcrystalline cellulose (Avicel® PH 200) and lactose for 5 minutes. The composition is shown in Table 5a.
3. Magnesium stearate was added to the powder blend, and the powder blend was mixed for an additional 3 minutes.
4. The powder blend was compressed (Manesty Beta tablet press) using the parameters shown in Table 5b.

The tablets were analyzed by pXRD, which showed that the Zolpidem hemitartrate remained in polymorph E.

TABLE 5a
Tablet Composition

| Components | Wt (g) |
|---|---|
| Zolpidem hemitartrate granules | 628 |
| Magnesium stearate | 4.28 |
| Microcrystalline cellulose (Avicel ® PH 200) | 50 |
| Lactose 316 (Farmost) | 150 |
| Total | 832.28 |

TABLE 5b
Tableting Parameters

| | |
|---|---|
| Tooling | Punches 0.1575 × 0.3957 |
| | Modified Oval (046423) |
| | Die (84091) |
| Tablet wt (mg) | 120 |
| Hardness | 6.7 kPa |
| Compression Force | 6.0 kN |
| Speed | 80 RPM |

### Example 7

### Preparation of Coated Placebo Tablets Having Zolpidem Hemitartrate in Polymorph E

Zolpidem hemitartrate having a mixture of several polymorphs (A, C, and D) was used to coat a placebo tablet (preparation described in Example 5) according to the following steps:
1. An active solution was prepared containing Zolpidem hemitartrate (80 g) and water (1700 g) by stirring for 1 hour.
2. Opadry® (34 g) was added to the active solution, and the solution was stirred for 1 hour. The components for preparing the tablets are shown in Table 6a.
3. Placebo tablets (600 g, each tablet weighing 122 mg) were charged into the pan coater and coated with the active solution of step 2 using the coating parameters described in Table 6b to obtain tablets having the composition shown in Table 6c.
4. The coated tablets were milled using CoMil at 1300 rpm with approximately 1000 μm grated screen (2A062G03123139) and impeller (2A1601173) to obtain granules.
5. The granules were blended with Avicel® (50 g) and lactose (150 g) and mixed for 5 minutes.
6. Magnesium stearate (4.28 g) was added, and the blend was mixed for an additional 3 minutes. The total composition of the blend is shown in Table 6d.
7. The blended material was pressed into tablets using a tablet press (Manesty Beta press) using the parameters shown in Table 6e.

The Zolpidem hemitartrate in the resulting coated tablet was found, by pXRD analysis, to be polymorph E.

TABLE 6a
Tablet Composition

| Components | Wt (g) | Note |
|---|---|---|
| Placebo tablets | 600 | |
| Zolpidem hemitartrate (A, C & D) | 80 | |
| Water | 1700 | |
| Opadry ® | 34 | 2% solution |

TABLE 6b
Coating Process Parameters
Target Process Parameters

| | |
|---|---|
| Inlet Air Temp | 100° C. |
| Exhaust Air | ~35-40° C. |
| Inlet Airflow | 31 CFM |
| Pan Speed | 20 rpm |
| Pump Speed | 15 rpm |
| Pan Charge | 600 g |
| Pump Reverse Time | 99 sec |
| Nozzle Pressure | 11 psi |
| Pan Jog Time | 10 sec |

TABLE 6c
Tablet Composition

| Components | Wt (g) | w/w % |
|---|---|---|
| Zolpidem Hemitartrate | 80 | 11.76% |
| Core tablet | 600 | 88.24% |
| Total | 680 | 100% |

TABLE 6d
Final Tablet Composition

| Components | Wt (g) |
|---|---|
| Zolpidem hemitartrate granules | 628 |
| Magnesium stearate | 4.28 |
| Avicel ® | 50 |
| Lactose 316 | 150 |
| Total | 832.28 |

TABLE 6e
Tableting Parameter

| | |
|---|---|
| Tooling | Punches 0.1575 × 0.3957 Modified |
| | Oval (046423) |
| | Die (84091) |
| Tablet wt (mg) | 120 |
| Hardness | 6.7 kPa |
| Compression Force | 6.0 |
| Compression force % RSD observed | 12-14% |
| Speed | 80 RPM |

In view of the above, it will be seen that the several objects of the invention are achieved and other advantageous results attained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense.

1. A method for converting a hemitartrate salt of a compound having the structure: 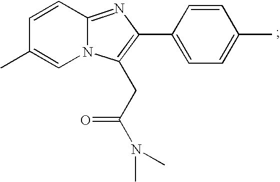
to a desired polymorph of the hemitartrate salt of the compound, the method comprising:
preparing a tablet comprising the hemitartrate salt of the compound; and
solvating the tablet with an amount of a solvent to convert the hemitartrate salt of the compound to the desired polymorph of the hemitartrate salt of the compound.

2. The method of claim 1 wherein the solvent is a liquid or a vapor.

3. The method of claim 2 wherein the solvent is water vapor.

4. The method of claim 2 wherein the solvent is liquid water.

5. The method of claim 4 wherein the tablet absorbs water such that water comprises at least about 5% by wt. of the tablet.

6. The method of claim 5 wherein the desired polymorph is polymorph E.

7. The method of claim 3 wherein the hemitartrate salt of the compound is solvated by exposing the hemitartrate salt of the compound to an atmosphere comprising water vapor having a relative humidity of greater than about 50%.

8. The method of claim 7 additionally comprising heating the tablet to at least about 40° C.

9. The method of claim 8 wherein the desired polymorph is polymorph D.

10. The method of claim 9 wherein the tablet additionally comprises excipients selected from the group consisting of lactose, fructose, maltodextrin, maltose, mannitol, sorbitol, sucrose, citric acid, tartaric acid, glycolic acid, acetate, citrate, tartrate, oxalate, phosphate, carbonate, microcrystalline cellulose, hydroxymethylpropyl cellulose, ethyl cellulose, propyl cellulose, starch, sodium starch glycolate, magnesium stearate, and combinations thereof.

11. The method of a claim 10 wherein the tablet additionally comprises lactose, magnesium stearate, starch, sodium starch glycolate, and microcrystalline cellulose.

12. A method for converting a hemitartrate salt of a compound having the structure: 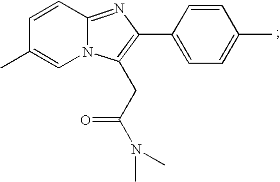
to a desired polymorph of the hemitartrate salt of the compound, the method comprising:
preparing a tablet comprising the hemitartrate salt of the compound; and
heating the tablet to convert the hemitartrate salt of the compound to the desired polymorph of the hemitartrate salt of the compound.

13. The method of 12 wherein the tablet is heated to at least about 50° C.

14. The method of claim 13 wherein the tablet additionally comprises excipients selected from the group consisting of lactose, fructose, maltodextrin, maltose, mannitol, sorbitol, sucrose, citric acid, tartaric acid, glycolic acid, acetate, citrate, tartrate, oxalate, phosphate, carbonate, microcrystalline cellulose, hydroxymethyipropyl cellulose, ethyl cellulose, propyl cellulose, starch, sodium starch glycolate, magnesium stearate, and combinations thereof.

15. The method of claim 13 wherein the tablet comprises lactose, magnesium stearate, and hydroxymethylpropyl cellulose.

16. The method of claim 13 wherein the tablet comprises lactose, cellulose, and magnesium stearate.

17. The method of claim 14 wherein the desired polymorph is polymorph C.

18. A method for converting a hemitartrate salt of a compound having the structure. 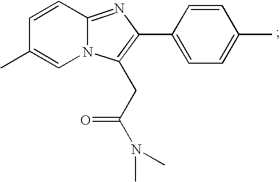
to a desired polymorph of the hemitartrate salt of the compound, the method comprising:
preparing a coating solution comprising the hemitartrate salt of the compound; and
coating a tablet with the coating solution to convert the hemitartrate salt of the compound to the desired polymorph of the hemitartrate salt of the compound.

19. The method of claim 18 wherein the coating solution is prepared by dissolving the hemitartrate salt of the compound in liquid water.

20. The method of claim 19 wherein the coating solution further comprises hydroxymethylpropylcellulose.

21. The method of claim 20 wherein the tablet comprises lactose, microcrystalline cellulose, magnesium stearate, hydroxymethylpropylcellulose, and sodium starch glycoate.

22. The method of claim 20 wherein the tablet comprises lactose, microcrystalline cellulose, and magnesium stearate.

23. The method of claim 22 wherein the tablet is coated by spraying the coating solution on the tablet.

24. The method of claim 23 wherein the desired polymorph is polymorph E.
